(19) United States
(12) Patent Application Publication
(10) Pub. No.: US 2017/0362615 A1
(43) Pub. Date: Dec. 21, 2017

(54) METHOD FOR PREPARING ORGANIC COMPOUNDS

(71) Applicants: HELMHOLTZ-ZENTRUM FUR UMWELTFORSCHUNG GMBH - UFZ, Leipzig (DE); DBFZ DEUTSCHES BIOMASSEFORSCHUNGSZENTRUM GEMEINNUTZIGE GMBH, Leipzig (DE)

(72) Inventors: Falk HARNISCH, Leipzig (DE); Luis Felipe MORGADO ROSA, Leipzig (DE); Heike STRAUBER, Leipzig (DE); Sabine KLEINSTEUBER,, Taucha (DE); Michael DITTRICH-ZECHENDORF, Leipzig (DE); Tatiane Regina DOS SANTOS, Braunschweig (DE); Uwe SCHRODER, Braunschweig (DE)

(21) Appl. No.: 15/326,007

(22) PCT Filed: Jul. 10, 2015
(86) PCT No.: PCT/EP2015/065877

§ 371 Date: Aug. 25, 2017

(30) Foreign Application Priority Data
Jul. 24, 2014 (DE) 10 2014 214 582.1

(51) Int. Cl. C12P 7/54; C12P 7/52; C12P 5/02; C12P 7/40; C25B 15/02; C25B 3/02

(52) U.S. Cl. CPC C12P 7/54; C25B 15/02; C25B 3/02; C12P 5/023; C12P 7/40; C12P 7/52

(57) ABSTRACT

The invention relates to a method for preparing organic compounds with recovery of product liquids, which comprise short-chain and medium length-chain carboxylic acids having a chain length of from 2 to 16 carbon atoms, by anaerobic fermentation of biomass with mixed microorganism cultures with suppression of methane formation and by electrolytic treatment of these product liquids containing the carboxylic acids with a constant or varying oxidation flow for the recovery and isolation of the target compounds.

The invention relates to a method for preparing organic compounds by fermenting biomass and by subsequent electrolytic treatment.

Hydrocarbon compounds such as alkanes, alkenes and others, in particular the organic basic chemicals ethylene, propylene and 1,3-butadiene and aromatic compounds such as phenol, are of great industrial relevance and are preferably obtained by petrochemical methods from fossil raw materials such as crude oil and natural gas. This applies to the hydrocarbons and especially mixtures thereof which are obtained by refining. Depending on the mixing ratio and chain length of the hydrocarbons, the different fractions are classified according to their boiling point.

Also known is the electrochemical reaction of organic acids by means of what is known as the Kolbe reaction (Kolbe, H., Justus Liebigs Ann. Chem., 1849, 69, 257-294) in order to obtain alkanes. Kolbe electrolysis is one of the oldest known electro-organic reactions. Since then, the decarboxylation of various natural carboxylic acids (Cₙ—COOH) in aqueous and organic media (electrolyte solutions) such as methanol, acetonitrile, etc. on different electrode materials such as Pt, Ti or platinised stainless steel has already been described frequently in the literature. In this regard there are several overview articles, including H. J. Schäfer, Top. Curr. Chem., 1990, 152, 91-151. The processes are carried out under extreme pH conditions or with high salt concentrations or preferably with use of organic solvents or ionic liquids.

Furthermore, U.S. Pat. No. 8,241,881 B2 describes a method for preparing hexane from fermentable sugars. The sugars are fermented with use of pure bacteria cultures or yeasts, which predominantly form butyric acid. The formed butyric acid is subjected to Kolbe electrolysis or photo-Kolbe electrolysis in order to yield hexane. The fermentable sugars originate from lignocellulose materials, such as wood products, switch grass, or agricultural waste products.

The electrochemical conversion in a culture medium or in the presence of microorganisms, however, poses a huge challenge, since side reactions occur to an increased extent in complex media and
can lead to a (potential) reduction of the production rates/yields,
(insofar as the process is performed in a reactor or a closed liquid flow) can lead to a “blocking” of the electrode surface and therefore to inactivation thereof,
(insofar as the process is performed in a reactor or a closed liquid flow) can lead to an undesirable (harmful) interaction with the microorganisms.

The object of the invention was to find alternative methods for preparing organic compounds which avoid the petrochemical method, known per se, of recovery from fossil raw materials such as crude oil and natural gas and which provide these products economically in good yields. In particular, the object of the invention was to develop a method which enables the storage and conversion of energy and also the sustainable recovery of hydrocarbon compounds from complex biomass.

This object could be achieved by a method which combines the fermentation of biomass and electrochemical treatment. The method according to the invention allows the preparation of medium-chain and long-chain alkanes and other hydrocarbon compounds and mixtures thereof from simple and complex biomasses by the combination of microbial fermentation and electrochemical oxidation. Organic compounds are preferably provided, which comprise C₆ to C₁₈ alkanes as main product, which are obtained possibly in mixture with corresponding derivatives, such as ethers, esters, alcohols, etc.

In the case of anaerobic fermentation, biomass of complex composition is broken down, as is known, by undefined mixed microorganism mixtures in unsterile conditions to form methane and carbon dioxide. Bacteria and archaea are involved in the process. Whereas the methane-forming step is catalysed exclusively by archaea, all other metabolic steps (hydrolysis, acidogenesis, acetogenesis) are carried out by bacteria.

The invention now makes use of the fact that with incomplete anaerobic fermentation up to methane formation, a wide range of fermentation products, particularly organic acids and alcohols and also hydrogen and carbon dioxide, are produced and in accordance with the invention can be used in an uncomplicated manner as starting products in a subsequent electrolytic method step.

For the acid production, all simple and complex, solid and liquid biomasses in principle are suitable in conjunction with mixed microorganism cultures, which can be of plant, animal or microbial origin and can also be used for biogas production. The used biomass can be selected from the groups of energy plants and residual materials and waste products from agriculture and industry. Extracts and processing products therefrom (for example sugars, celluloses), algae and yeasts can also be used as starting biomass. Gas mixtures resulting from the gassing of biomass or fossil raw materials such as coal (for example (bio)syngas, pyrolysis gas) are also suitable. Biomass which is optionally silaged is also suitable, for example corn silage, grass silage. Such lactic acid fermented substrates are used with preference for microbial chain lengthening for mono- or co-fermentation on account of their favourable chemical composition (high proportion of lactic acid). The biomass can also be pre-treated by means of other physical, physical-chemical, chemical and/or biological methods. Wood and products which are based predominantly on wood are less suited on account of the high degree of lignification.

The method according to the invention is characterised by fermentation of the biomass with suppression of methane formation for recovery of product liquids which comprise mixtures of short-chain and medium length-chain carboxylic acids having a chain length of from 2 to 6 carbon atoms, and by a subsequent electrolytic treatment of the product liquid containing the carboxylic acids in mixture with a constant or varying oxidation current for recovery of organic compounds. The term product liquid is understood to mean the liquid fraction which after fermentation of the biomass is enriched with the desired fermentation products, i.e. the mixture of short-chain and medium length-chain carboxylic acids

Insofar as solid biomass is used primarily, this is brought into contact for example with a liquid in preparation of the fermentation. It can be soaked in the liquid or mixed or sprinkled therewith, such that a fermentation broth is formed. For soaking/mixing/sprinkling, water or other liquids such as liquid manure and/or liquid fermentation residues can be used, which already have an at least mushy structure on account of their consistency and also can originate from a fermentation process. Biomasses that are already predominantly liquid can be used directly.

For fermentation, temperatures between 10 and 100° C. are selected in the fermenter. This can be implemented by heating the fermenter and/or by adding heated liquid. The resultant product liquid, which preferably contains at least 5 g/L of short-chain and medium length-chain carboxylic acids, is then treated electrolytically. Solid fermentation residues present after the fermentation are removed as necessary. The product liquid can be purified and/or concentrated prior to further electrolytic treatment.

During the fermentation, the pH value lies in a range of from 3.5 to 9.5. It adjusts itself in the process. A low pH value can be ensured for example by use of chemicals (addition of mineral acids). However, this is often unnecessary, since the organic acids created during the process generally reduce the pH value sufficiently. For fermentation, mixed cultures of acid-forming microorganisms can be added to the biomass. In order to ensure the lengthening of short-chain carboxylic acids as starting materials, energy-rich reduced substances such as alcohols, for example ethanol, 1-propanol, 2-propanol, and/or lactic acid can be added to the fermentation step. The necessary alcohols such as ethanol are formed as applicable as by-product (by alcoholic fermentation). Lactic acid is created for example as main product of the lactic acid fermentation, is also formed by anaerobic fermentation, and is contained in large concentrations for example in silaged biomass as starting substrate, such as corn silage or grass silage.

The acids are preferably present after fermentation of biomass in the form of a mixture of branched and/or unbranched mono, hydroxy, and/or dicarboxylic acids in the product liquid. They are preferably carboxylic acids having 4 to 10 carbon atoms.

In particular, they are mixtures which preferably comprise high concentrations of n-butyric acid, iso-butyric acid, n-valeric acid, iso-valeric acid, n-caproic acid, n-heptanoic acid and n-caprylic acid.

The contained carboxylic acids can be determined by various methods, such as gas chromatography (GC) or liquid chromatography (HPLC).

The acid formation in the fermenter can be stimulated by different measures known to a person skilled in the art. These include, primarily, the measures that are used to prevent methane formation. One method for example lies in minimising the residence time of substrate in the reactor (hours to a few days, preferably at most 5 days). This causes microorganisms having long generation times, such as methane-forming archaea, to grow. A further possibility lies in carrying out the process at a low pH value (Jiang, J., Zhang, Y., Li, K., Wang, Q., Gong, C., Li, M. 2013. Volatile fatty acids production from food waste: Effects of pH, temperature, and organic loading rate. Bioresource Technology, 143, 525-530). In biogas processes used in the field of biotechnology, an acidification generally leads to an irreversible inhibition of methane production. Acid-forming bacteria, by contrast, tolerate such pH values or even have their growth optimum in this range.

A further measure for stimulating the formation of organic acids from biomass lies in a pre-treatment of the mixed microorganism culture (inoculum) which will be used for the anaerobic fermentation. This can be exposed to high temperatures (autoclaving, heat shock), or chemicals (methane-formation inhibitors, such as 2-bromethanesulfonic acid or fluoromethane as specific inhibitors of methanogenesis) can be added (both to the inoculum and to the fermentation broth) in order to inactivate methane-forming microorganisms. Specific acid-forming bacteria, particularly spore formers, survive the heat treatment and germinate again in favourable ambient conditions. For the production of carboxylic acids having a chain length Cₓ with x>4, the setting of the pH value to between 5.0 and 8.0 is advantageous. For the microbial anaerobic production of carboxylic acids having a carbon chain length Cₓ with x≧5, two biochemical mechanisms should be highlighted:
i) the breakdown of long-chain carboxylic acids by β-oxidation during the fermentation of fat-rich substrates, such as slaughterhouse waste, used greases from grease traps, or plant oils, or
ii) the formation of short-chain fatty acids by microbial chain lengthening (reverse β-oxidation). In the case of microbial chain lengthening, the chain lengths of the carboxylic acids are lengthened in each case by C₂ units, for example acetic acid becomes n-butyric acid, n-butyric acid becomes n-caproic acid, n-caproic acid becomes n-caprylic acid, and propionic acid becomes n-valeric acid (Steinbusch, K. J. J., Hamelers, H. V. M., Plugge, C. M., Buisman, C. J. N. 2011. Biological formation of caproate and caprylate from acetate: fuel and chemical production from low grade biomass. Energy & Environmental Science, 4(1), 216).

In principle, the anaerobic fermentation for acid production can be performed in any type of fermenter as have become established for biogas production. These include percolation methods, but for example also stirrer tanks, UASB (Upflow Anaerobic Sludge Blanket) reactors, ASBR (Anaerobic Sequencing Batch Reactors) or plug flow vessels for anaerobic fermentation.

The percolation method, which is preferably used in the method according to the invention, will be described in greater detail hereinafter. Solid substrate (the used biomass) is sprinkled from above with a liquid (preferably water which has been inoculated with liquid fermentation residue from another fermentation process) in order to form the fermentation broth, the liquid is caught again beneath the substrate, is collected in a storage container, and is pumped again within a circuit above the substrate (biomass). The temperature in the fermenter is preferably between 10 and 100° C. (in a psychrophilic process <30° C., in a mesophilic process 30-45° C., and in a thermophilic process 45-60° C. Hyperthermophilic processes at >60° C. are also possible). The temperature can be ensured by heating the fermenter content and/or the liquid (percolate). Since there is no stirring system provided, solids and liquid are mixed by intense pumping of the percolate and spraying of the substrate. The liquid can be easily separated from the solids, and there is generally no need for a separate solid-liquid separation step. The reactor chamber also contains no moving parts. This method is thus insensitive to mechanical impurities.

Percolation methods can be performed in batch operation (filling the fermenter with substrate, fermentation, and removal of the solid fermentation residue and the product liquid with the acids, then renewed filling, etc.) or in semi-continuous operation. In the case of semi-continuous operation, a number of fermenters are connected in series, are each started up in batch operation at different times, and all fermenters are sprinkled with the same percolation liquid. In this way, an inoculation of fresh biomass (substrate) with acid-forming microorganisms can be generated, as well as a temporally uniform product formation in the product liquid. The product liquid created with the percolation method can be stored for a number of days without significant quality loss (i.e. no breakdown or only slight breakdown of the acids) on account of its chemical stability (low pH value).

As already mentioned above, the anaerobic fermentation for acid production can be carried out in principle in any type of fermenter as have become established for biogas production. These also include arrangements for biogas production which use separate fermenters for hydrolysis/acid formation and acetic acid/methane formation, wherein the product liquid from the first-phase reactor, i.e. the fermenter for hydrolysis/acid formation, is electrolytically treated. If solid substrates and liquid are mixed to form a fermentation medium (no process-inherent solid-liquid separation), a separate process step can be carried out after the anaerobic fermentation for solid-liquid separation. Various techniques can be used for this purpose which are already established on the market, such as cross-flow filtration, screw press separator, decanter, band filter, or curved sieve.

After the fermentation of the biomass, a homogeneous product liquid is provided which can be easily handled, can be pumped, is enriched with the desired fermentation products, i.e. the mixture of short-chain to medium length-chain (C₂ to C₁₆) carboxylic acids, and easily can be made accessible to the second method step for electrochemical conversion. Methods which ensure an integrated concentration/in situ separation of the carboxylic acids as appropriate are known in the literature (Agler M. T., Spirito C. M., Usack J. G., Werner J. J. and Angenent L. T. (2014). Development of a highly specific and productive process for n-caproic acid production: applying lessons from methanogenic microbiomes. Water Science and Technology, 69(1), 62-68). The longer is the chain of the carboxylic acids, the more hydrophobic (less water-soluble) they are. n-caproic acid for example is only water-soluble up to a concentration of 10.19 g/L. This property makes it possible to remove medium length-chain undissociated carboxylic acids from the fermentation medium by means of hydrophobic solvent (for example via hollow fibre membrane), continuously and during running operation. In addition, a product inhibition of the acid-forming microorganisms is thus avoided, which in turn leads to an increase in the yield.

As already mentioned, solid fermentation residues that are created can be separated as appropriate and further treated in a separate utilisation step. Another possibility lies in dispensing with a solid-liquid separation after fermentation and using the fermentation media enriched with the organic acids, i.e. the product liquid comprising the carboxylic acids, directly for electrochemical conversion, without separation of the solids. In other words, fermentation and electrolytic treatment can take place directly in the fermenter, or the product liquid is transferred into another container for electrolytic treatment.

In accordance with the invention the subsequent electrolytic treatment is carried out with a constant positive oxidation current (galvanostatic operation) or with a varying oxidation current. The product liquid is advantageously treated with bases or acid prior to the electrolytic treatment in order to modify the pH value. A pH value ranging from 5.5 to 11 is preferred for the electrolytic treatment.

Galvanostatic operating modes result in a corresponding potential at the electrode. The current flow is preferably specified as current density in relation to the geometric surface (in mA/cm²) or in relation to the reactor volume (in mA L⁻¹). A galvanostatic operating mode is usually preferably selected. In particular, “pulse methods” can also prove to be advantageous. A pulsed current supply (also referred to as varying oxidation current) can be used, in which case the current alternates between two values, of which one is smaller than the other, can be zero, or can even have reversed polarity. In the pulse method, the current flow (working current flow) is altered to another current flow (“pulse current”) in constant or alternating time intervals. This has the advantage that a deactivation or blocking of the anode is prevented. The phases of current flow (production) and no current flow (no production) alternate with one another, wherein the pulse duration varies between 1 second and 2 days, but is always shorter than the duration of the application of the working current flow.

Various metals and non-metals can be used as anode materials. For example, platinum, titanium, etc. and the binary, trinary and higher alloys thereof as well as boron-doped diamond electrodes are used as metals. Furthermore, electrode materials which are based on a functional surface coating of the specified materials on a conductive carrier material, also including metal materials such as stainless steels or non-metal materials such as graphites, are included. By way of example, graphite and graphite modifications (including carbon nanotubes or carbon nanoparticles) and also all corresponding composite materials can be used as non-metals.

The electrode specification can include all geometric shapes and modifications of the aforementioned metals and non-metals, in particular sheets, plates, films, rods, tubes, sponges, nonwovens, woven fabrics, brushes, cylinders.

After electrolytic treatment in accordance with the invention, organic compounds are obtained which preferably comprise C₆ to C₁₈ alkanes as main products. They are recovered in a mixture with corresponding derivatives such as ethers, esters, alcohols, etc. as applicable. These products are deposited as second phase in aqueous solution on account of their low solubility and can thus be easily separated, however an extraction from the aqueous reaction solution by means of centrifugation or membrane methods, which are known to a person skilled in the art, can be performed alternatively or in addition.

Since the electrochemical reaction can be carried out in aqueous solutions, the separation of the organic product (during the reaction) from the aqueous reaction solution makes it possible to isolate the product very easily and to recycle the aqueous electrolyte solution, and thus allows the entire method to be carried out in a continuous process.

The method according to the invention has a series of further advantages:

The anaerobic fermentation of organic biomass can be carried out, depending on the substrate to be fermented, in reactors of different construction. Liquid or solid fermentation systems, such as stirrer tanks, plug flow vessels, or box fermenters are standard. Here, both batch methods and (semi) continuous methods are possible. The described method can thus be adapted for a large number of reactors and applications.

The method can be carried out on different scales and therefore can also be very effectively decentralised.

The method requires only small amounts of electrical energy (direct current). It is therefore outstandingly suitable for coupling with alternative and decentralised methods for generating electrical energy, for example photovoltaics or wind power.

Due to the possibility of eliminating the carboxylic acids from the reactor as the process is being carried out, i.e. due to the prevention of a potential product inhibition, higher carboxylic acid yields can be achieved.

The method leads to a mixture of hydrocarbon compounds (alkanes, ethers, alcohols) and is therefore suitable both for the production of potential alternative fuels, specifically in particular with regard to the heating value and the boiling point curves and basic chemicals.

Furthermore, the solid and liquid (depending on the substrate and method) fermentation residues and hydrolysis gas created during the anaerobic fermentation besides the organic acids can be used for biogas production. In this case, the energy obtained from the biogas can be used directly as process energy for the anaerobic fermentation (electrical energy for pumps or stirrers, thermal energy for the heating of the reactors) or electrical energy can be used directly for electrochemical conversion of the acids.

Both the fermentation residues resulting from the biogas process and the fermentation residues from the acid production can be recycled as fertiliser or can be processed to form compost (closed material circuits).

The gas created during the anaerobic fermentation primarily contains hydrogen and carbon dioxide (what is known as hydrolysis gas). This gas, depending on its hydrogen content, can also be burned directly for energy production or can be added to the biogas from the fermentation residue utilisation. Alternatively it is expedient in some circumstances to introduce the hydrogen-carbon dioxide mixture into a (biogas) reactor and to convert this into methane (methanogenesis).Alternatively, a combination of the anodic Kolbe reaction with a cathodic reduction reaction, which stimulates microbial chain lengthening (see above) (electrochemical microbiome shaping), is possible.

Liquids from the electrochemical conversion which are loaded with alkane traces, can also be re-used in the method.

This residual liquid can be used either as process liquid for the aerobic fermentation or can be added to this process liquid (recirculation). However, in this case it must be ensured that the microorganisms are adapted to the specific conditions of the alkane loading. Alternatively, the residues can be methanised in a biogas process. Alternatively, the residues can be methanised in a biogas process. Here as well, the microorganisms must be adapted to the alkane loading.

The storage capability of the product liquid with the carboxylic acid mixture after fermentation is of particular importance. It offers the possibility to operate the electrochemical conversion as required, for example whenever current is inexpensive (for example current overproduction from photovoltaics/wind) or the further possibility to produce carboxylic acids “in advance”.

In addition, the storage capability of the percolate from the percolation method used with preference enables a combination of batch method (anaerobic fermentation) and continuous process (electrochemical conversion).

Even a physical separation of an aerobic fermentation and electrochemical conversion step is possible, since the acids can be transported over longer distances. However, in this case a concentration of the acids in the percolate for volume reduction is advantageous. For example, this can be a separate extraction step.

Apart from the energy-intensive biomass gassing and generation of synthesis gas and subsequent Fischer-Tropsch synthesis, there are currently no methods for converting complex biomasses into (mixtures) of hydrocarbon compounds.

The method according to the invention makes it possible to convert complex biomass into (mixtures of) hydrocarbons. Complex biomasses and/or electrical energy from sustainable sources can thus be converted into products that are valuable in terms of energy and that can be stored. The method also can be carried out in a decentralised manner and can be integrated into existing infrastructures. It also can be carried out independently of electrical infrastructure (operation with decentralised electrical energy sources, such as photovoltaics or wind turbines). The products can be used both as basic/fine chemicals and as alternative fuels.

The invention will be explained in greater detail hereinafter on the basis of examples.

### PRACTICAL EXAMPLES

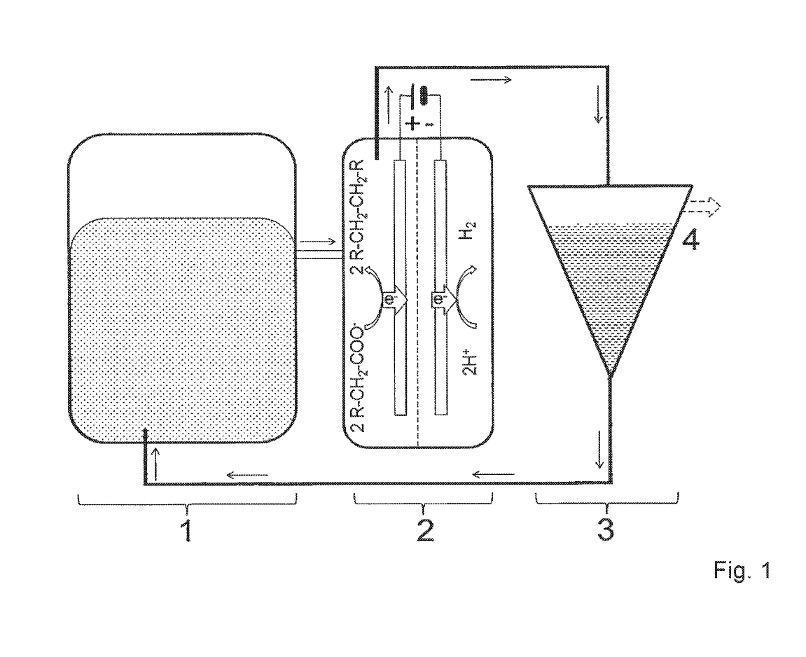

FIG. 1: sequential execution of microbial acid production and electrochemical acid oxidation;
1: fermenter, 2: electrochemical reactor, 3: product separation, 4: (organic) product phase

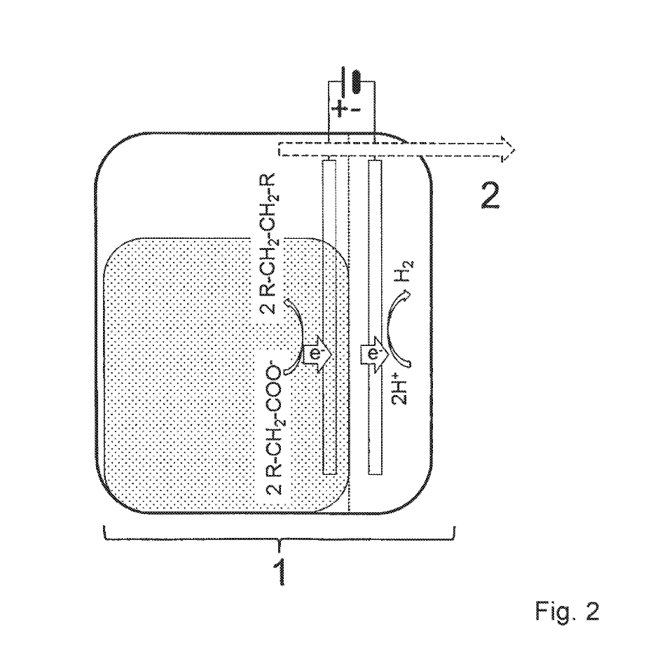

FIG. 2: in situ execution of microbial acid production and electrochemical acid oxidation.
1: fermentation with in situ electrochemical reaction, 2: (organic) product phase

(Note: in FIG. 1 and FIG. 2, the hydrogen development reaction is always illustrated as cathode reaction, see the main text with regard to the use thereof and alternative reduction reactions).

### Example 1

Anaerobic fermentation of corn silage in a percolation method (batch test):

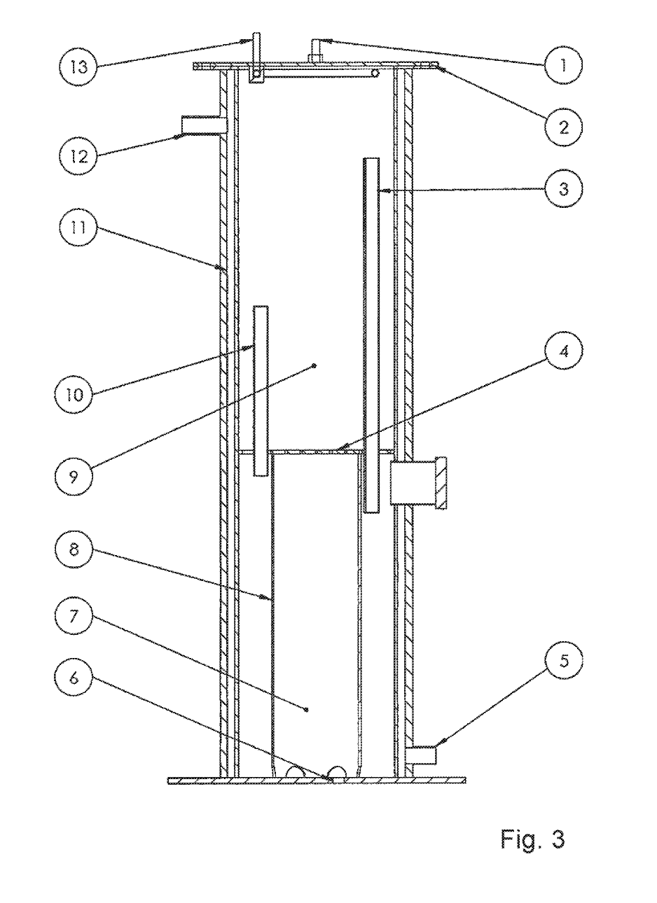

### Test Structure (FIG. 3)

1 biogas removal
2 fermenter lid with percolation ring system
3 pressure compensation line
4 supported sieve
5 heating line drain
6 product harvesting and circulation ring line drain
7 percolate storage chamber
8 support pipe for supported sieve
9 solids fermentation chamber
10 overflow
11 double-walled fermenter jacket
12 heating line feed
13 circulation ring line feed

A PVC double-walled reactor divided into two compartments (total volume 45 L) with thermostat temperature control (38° C.) and an integrated sprinkler system was constructed (FIG. 3). A sieve bottom (hole diameter 2 mm) was used to retain solid substrate constituents and thus separated the upper reactor chamber, into which substrate was filled, from the lower part, in which the percolate was caught. Two pipes, which connected the compartments to one another, additionally belonged to the internal fittings of the reactor. One of the pipes served for pressure compensation between the two compartments. The other constituted a drain safeguard pipe, which in the event of a blockage of the sieve bottom prevented an overflow of the percolate in the upper compartment. A pump was used to circulate the percolate from the lower reactor region to the sprinkler apparatus below the reactor lid. A temperature-controlled water bath was used to heat the reactor via the double-walled system.

### Execution

2 kg_{fresh mass} of corn silage were mixed with 15 g Mn(OH)₂ and filled into the reactor. 5 kg of deionised water were used as a basis for the fermentation broth and were added via the corn silage. Then, 1 kg of inoculum liquid (process liquid from a two-stage biogas facility) was added via the corn silage. The percolate was caught and collected within the reactor beneath the sieve bottom. The reactor was closed and a tightness test was carried out (5 mbar N₂ overpressure).

Then, the pump was activated and the substrate was sprinkled for 15 min long with the percolate. The percolation by the peristaltic pump then followed in interval operation with a rate of 300 mL/30 min until the end of the test.

### Analytics

The percolate was tested at regular intervals for the qualitative analysis thereof. Percolate samples were removed from the circulation line via a drain port. Prior to the analysis, the samples were centrifuged (Megafuge 16R, Nereus, 10,000×g, 10° C., 10 min) and the pellet was separated from the supernatant. The concentrations of acetic acid, propionic acid, iso-butyric acid, n-butyric acid, iso-valeric acid, n-valeric acid, and n-caproic acid in the percolate were determined by means of gas chromatography (for method details see Example 2).

### Results

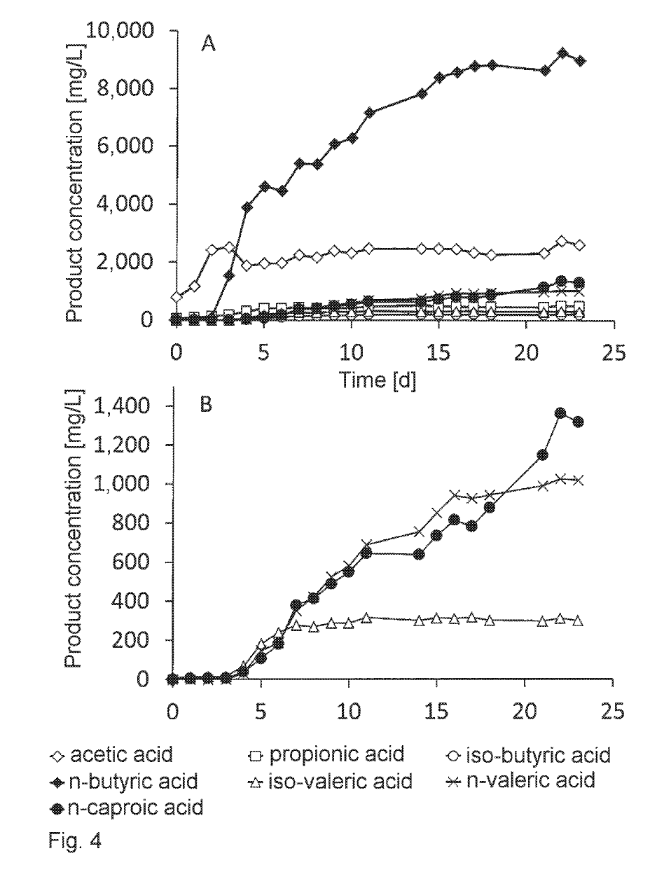

FIG. 4 shows the production of all measured organic acids (A) and, shown only in part, of C5 to C6 acids (B), specifically of n- and iso-valeric acid and of n-caproic acid in the course of the process.

Besides these acids, n-butyric acid and acetic acid were also created in significant amounts, respectively 8990 mg/L and 2620 mg/L. Further acids were formed in small amounts: ≈500 mg/L propionic acid, ≈200 mg/L iso-butyric acid. Heptanoic acid was not detected.

### Example 2

Continuous anaerobic fermentation of biogas waste in a two-phase method (hydrolysis/acid formation separate from acetic acid formation/methane formation)

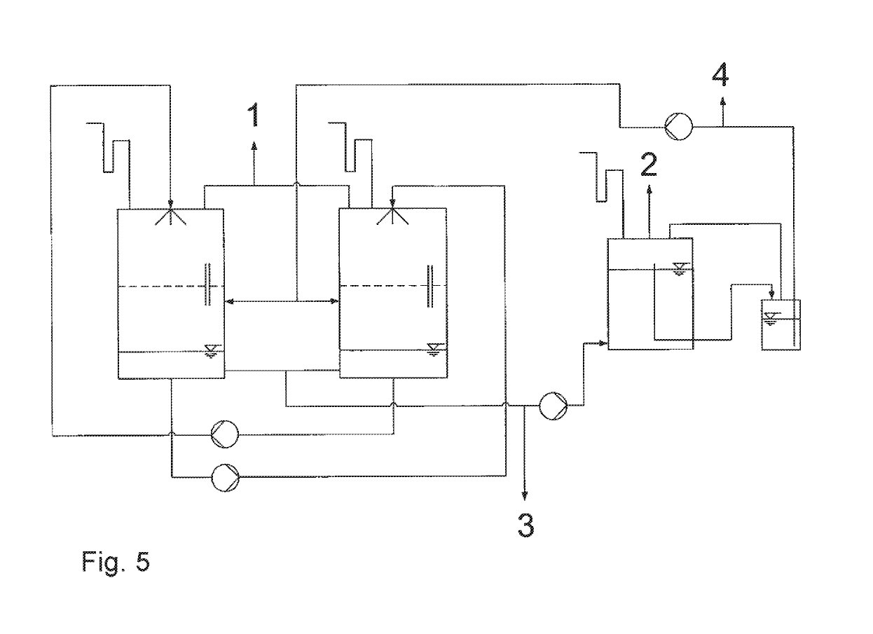

### Test Structure (FIG. 5):

1 Sampling port gas chamber 1^{st} stage
2 Sampling port gas chamber 2^{nd} stage
3 Percolate drain 1^{st} stage
4 Recirculation of fermentation residue from the 2^{nd} into the 1^{st} stage

The fermentation of biowaste was carried out in a two-phase reactor consisting of solid fermentation in a percolation method (hydrolysis+acid formation) and a stirred reactor (acetogenesis+methanogenesis). This test was performed as a double test, i.e. there were two two-phase reactor systems completely separated from one another, which here are named reactor systems 1 and 2. The first-phase reactors of systems 1 and 2 each consisted of two sieve bottom reactors coupled to one another as described in Example 1. The second-phase stirred reactors each had a working volume of 11 L and were filled with filling material formed from polyethylene as growth material for microorganisms. These reactors were provided with an overflow. The drains of the methane stages were fed back into the corresponding hydrolysis stages.

### Execution

The sieve bottom reactors were operated as percolation reactors as described in Example 1. Here, approximately 900 mL of the liquid phase of a reactor were pumped into the coupled reactor every half an hour. Approximately 2000 mL were pumped daily into the second-phase reactor from the percolate of the sieve bottom reactors.

Communal biowaste that had been removed from a composting plant on 26.03.2014 was used as substrate. At the start of the test, the percolation reactors were each loaded with 10.0 kg water, 4.0 kg biowaste, and 2.0 kg inoculum (drain of the hydrolysis stage of a two-stage biogas facility). The reactors were flushed with nitrogen, closed in an airtight manner, and the percolation was started.

The two sieve bottom reactors were charged twice weekly in alternation with 3.0 kg fresh biowaste. In order to compensate for the volume loss by sample removal, an additional 500 mL water were added every 2 weeks. After each substrate change, the reactors were flushed with nitrogen. The sampling was performed at least twice weekly, in each case on the day following the substrate change.

### Analytics

The pH value of the percolate was measured using a WTW pH 3310 pH electrode. Percolate samples were centrifuged by means of a Heraeus Megafuge 16R (10 min at 10,000×g and 10° C.) and the supernatant was examined by means of GC with regard to the concentrations of organic acids and alcohols (triple determination). For this purpose, 3.00 mL of supernatant were pipetted in each case into a 20 mL Headspace vial, mixed with 1.00 mL of a solution of the internal standard (2-methylbutyric acid; 187 mg/L), 0.50 methanol and also 2.50 mL of diluted sulphuric acid (1:4; (v/v)), and closed in a gas-tight manner. The separation was performed on an Agilent Tech. 7890A GC system on a ZB-FFAP (Phenomenex) column. The quantification was performed on the basis of calibrations and the internal standard. The volume of the formed gas of the hydrolysis stage was detected by means of a Ritter MGC-1 V3.1 PMMA milligas counter. The gas was caught in gas-tight bags made of aluminium PE composite foil. An analysis of the main constituents of the gas by means of GC was performed. For this purpose, 20 mL gas vials were closed in a gas-tight manner and were flushed with argon. In each case 1.00 mL of hydrolysis gas was removed by means of a syringe through a septum in the gas line prior to the MGC and was injected into the vials filled with argon. These samples were separated into the individual gas constituents and detected on a Perkin Elmer Clarus® 580GC on a Hayesep N and a Mole sieve 13×, ASAG column.

### Results

In the example, only the process data from the first-phase reaction are shown.

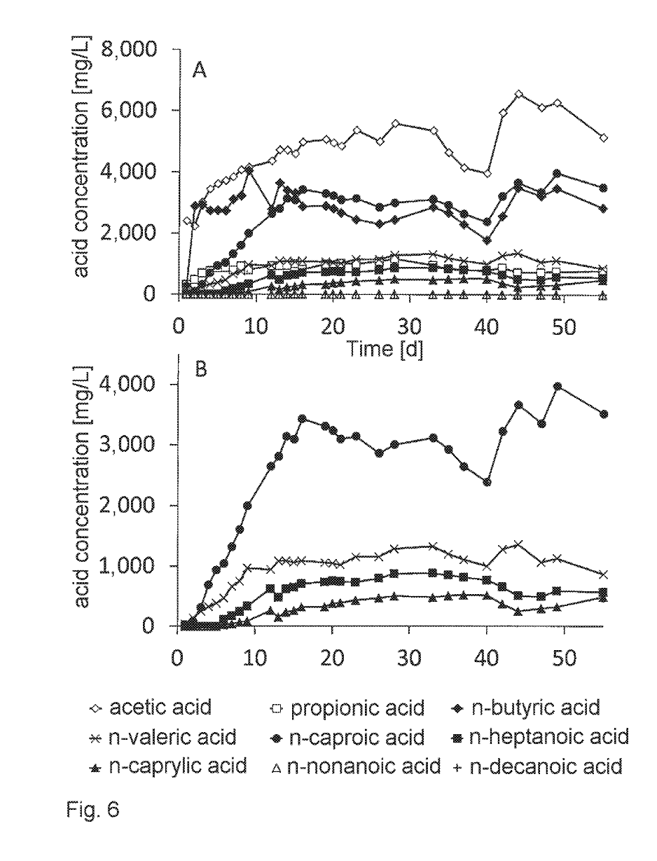

FIG. 6: production of unbranched organic acids (A) and, shown only in part, of unbranched C₅ to C₈ acids (B) in the first-phase reactor of the reactor system 2.

In addition, the concentrations of further acids and alcohols were detected (Table 1).

TABLE 1
Concentration of further acids and alcohols in the first-phase
reactors of the reactor systems 1 and 2

| | Maximum concentration |
|---|---|
| Acids: | |
| formic acid | <15 mg/L |
| iso-butyric acid | ≦300 mg/L |
| iso-valeric acid | ≦150 mg/L |
| iso-caproic acid | ≦25 mg/L |
| lactic acid | at the start 4300 or 3850 mg/L, later <1000 mg/L |
| Alcohols: | |
| ethanol | <1000 mg/L |
| 1-propanol | <350 mg/L |
| 2-propanol | <15 mg/L |
| 1-butanol | <100 mg/L |
| 2-butanol | <100 mg/L |

### Example 3

Electrochemical conversion of a carboxylic acid mixture in a batch test

### Test Structure

A mixture of carboxylic acids (see substrate) was set to a pH value of 5.5 using 60% potassium carbonate solution. The tests provided the described carbonate acid solution in 250 mL four-neck round-bottom flasks with 100 mL filling volume. Platinum (Goodfellow, Germany) having a geometric surface of approximately 2.7 cm² was used as working electrode. A platinum electrode with approximately 4 cm² was used as counter electrode, and an Ag/AgCl (sat. KCl) electrode (0.197 mV vs. SHE, SE10 type Meinsberg) was used as reference electrode. In addition to the electrode terminals, a sampling port and waste air cooling means were connected to the piston. The waste air was cooled by means of a Dimroth cooler, water-cooled to 4° C. A magnetic stirrer (4.5×14.5 mm) was used to continuously mix the solution at 500 rpm.

### Execution

Before the test was started, the dissolved oxygen of the carboxylic acid solution was displaced from the system by a 15-minute nitrogen flushing. The galvanostatically performed electrochemical synthesis lasting for 7 h with a current density (relative to the anode surface) of 130 mA/cm² was then started. Both the voltage between working electrode and counter electrode and between working electrode and reference electrode were recorded for control purposes. A sample of the aqueous phase was taken every hour (sample volume 400 μL for pH check and HPLC analytics). At the end of the test, in addition to the HPLC check, the organic phase was also removed and measured by means of GC-MS).

### Substrate

39 g/L n-butyric acid, 20 g/L n-valeric acid and 9 g/L n-caproic acid in distilled water were used as substrate.

### Analytics

The sample of the aqueous phase was used on the one hand to check the pH value by means of test strips (pH indicator rods 4.0-7.0, non-bleeding, Merck; pH indicator rods 7.5-14 non-bleeding, Merck).

On the other hand, an HPLC (high performance liquid chromatography) analysis (Shimadzu Corporation) enabled quantification of the content of carboxylic acids and water-soluble primary and secondary alcohols. A Hi-Plex H column (Agilent Technologies) was used for the separation at an oven temperature of 65° C., and a refraction index detector (RID-10A) was used for the detection. 5 mM sulphuric acid in water at a flow rate of 0.6 mL/min was used as mobile solvent. The substances were allocated into corresponding dilutions by their retention time on the basis of previous measurements of standard solutions and were quantified on the basis of the calibration of associated peak areas (R²=0.99).

GC-MS analytics (gas chromatography mass spectroscopy: gas chromatograph 7890A with column oven and mass spectrometer 5975 C inert MSD with Triple-Axix Detector Agilent Technologies) served for the qualitative and quantitative determination of alkanes, esters, alcohols and further by-products. The used capillary column (HP-SMS, 30 m length, 0.25 mm diameter and 0.25 μm film thickness, Agilent Technologies) was operated with the carrier gas helium 5.0 with a split of 0.1 mL/min. The measurements started at 35° C. with a holding time of 20 min, then the temperature was raised by 5 K/m in to 200° C. A further temperature rise to 300° C. was achieved with 30 K/m in and was then maintained for 2 min. The obtained peaks were compared with the mass spectral library (NIST 2008 Mass Spectral Library, G1033 A, Revision Jan 2008, 597× MSD, 7000A Triple Quad, Agilent Technologies) and calibrated with standards as necessary. The obtained samples were diluted in acetone 1:100 to 1:1000.

### Results

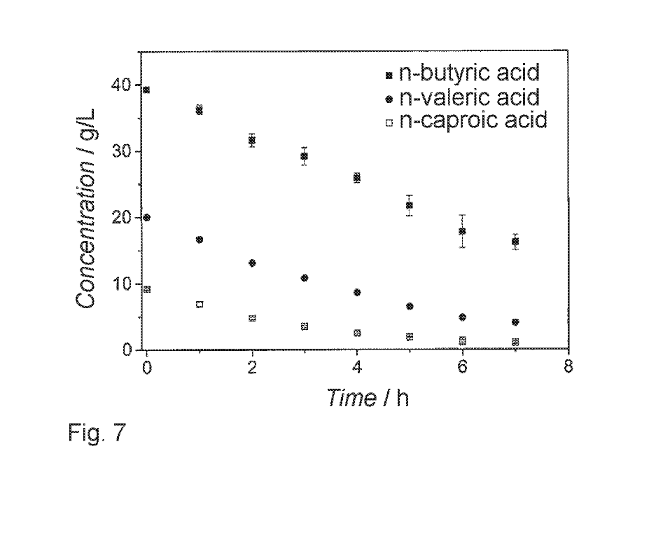

All used carboxylic acids were broken down and the following conversions were attained up to the end of the test (7 h): 59% n-butyric acid, 80% n-valeric acid, and 89% caproic acid. On the whole, a conversion of 77% of the used carboxylic acids could be achieved. FIG. 7 shows the change of the carboxylic acid concentration in Example 3 over the test time. The standard deviations are based on two identical tests.

The continuous formation of 1-propanol, 2-propanol, 1-butanol and 2-butanol was able to be detected by means of HPLC analytics. After 7 h of test running time, the following concentrations were provided respectively: 0.97 g/L, 2.77 g/L, 0.38 g/L and 1.86 g/L with a deviation of at most 3% within the test repetitions. It is possible that 1-pentanol and 2-pentanol were also formed in low concentrations below the corresponding detection limits.

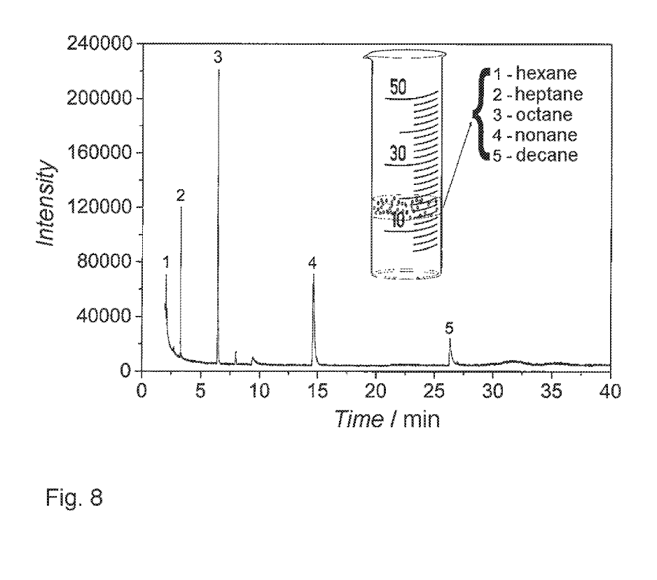

The GC-MS analytics of the organic phases diluted 1:1000 times in acetone showed exclusively representatives of the alkanes: n-heptane, n-octane, n-nonane and n-decane (see bottom of FIG. 8). In the 1:100 times dilution the reaction products stated in Table 2 could also be found. After the alkanes, esters were the second-largest product group, confirmed by alcohol formation during the electrochemical process.

The pH value of the carboxylic acid solution rose within the first two to three hours to 10.5 and remained for the rest of the test time constantly at 10.5.

The electron yield, that is to say Coulomb efficiency (CE), of 53% was calculated on the assumption that an individual electron was converted during the oxidation per converted acid molecule, and therefore exclusively the radical formation was considered.

TABLE 2
Main products of the organic phase from Example 3 with 1:100 times
dilution in acetone (basis: GC-MS analytics)

| Esters | Alkanes |
|---|---|
| butyric acid-1-methylethyl | hexane |
| ester | |
| butyric acid-1- | heptane |
| methylpropyl ester | |
| butyric acid-1-methylethyl | octane |
| ester | |
| butyric acid-1- | nonane |
| methylbutyl ester | |
| valeric acid-1- | decane |
| methylpropyl ester | |
| hexanoic acid-1- | |
| methylethyl ester | |
| valeric acid-1-butyl ester | |
| valeric acid-2-pentyl ester | |
| hexanoic acid-2- | |
| methylpropyl ester | |
| valeric acid-1-pentyl ester | |
| hexanoic acid-3-pentyl | |
| ester | |

FIG. 8:

Production separation on the basis of the example of the formation of n-octane from n-valeric acid and also GC-MS chromatogram with 1:1000 dilution of the organic phase in acetone (see analytics for details)

### Example 3a

### Conversion of Further Carboxylic Acids and Mixtures in a Batch Test

### Test Structure

see Example 3, for possible deviations see Table 3

### Substrate:

see Table 3

### Execution

see Example 3, for possible deviations see Table 3

### Analytics

see Example 3

Table 3 compares the obtained results of the various test approaches in summary. Here, n-valeric acid was examined in various concentrations and at various current densities relative to the anode surface (Table 3, no. 1-3). By way of supplementation, the electrochemical conversion of iso-valeric acid could be shown (Table 3, no. 4). In addition, a mixture of n-butyric acid and n-valeric acid was tested (Table 3, no. 5-6).

TABLE 3
Overview of different galvanostatically performed electrochemical tests with various carboxylic
acids in analogy to the test structure described in Example 4 (running time: 7 h)

| | | | | | | Conversion of | |
|---|---|---|---|---|---|---|---|
| | | Starting | Current | Conversion | | carboxylic | |
| | | concentration | density | in 7 h | CE | acids [mmol/ | |
| No | Substrate | [g/L] | [mA/cm²] | [%] | [%] | (cm² h)] | Main products organic phase |
| 1 | n-valeric acid^{#1} | 105 | | 23 | 82 | 0.768 | octane |
| | | | | | | | dibutyl peroxide |
| | | | | | | | valeric acid-1-methylpropyl ester |
| | | | | | | | valeric acid-butyl ester |
| 2 | n-valeric acid^{#1} | 102 | 150 | 60 | 69 | 1.939 | octane |
| | | | | | | | dibutyl peroxide |
| | | | | | | | valeric acid-1-methylpropyl ester |
| 3 | n-valeric acid | 39 | 130 | 85 | 35 | 0.975 | 2-hexanone |
| | | | | | | | octane |
| | | | | | | | dibutyl peroxide |
| | | | | | | | valeric acid-1-methylpropyl ester |
| | | | | | | | valeric acid-butyl ester |
| | | | | | | | valeric acid-pentyl ester |
| 4 | iso-valeric acid | 41 | 130 | 86 | 37 | 0.903 | ethyl hexane |
| | | | | | | | valeric acid-2methylpropyl ester |
| | | | | | | | valeric acid-butyl ester |
| 5 | n-butyric acid/n- | 25/26 | 100 | 56/76 | 50 | 0.855/1.014 | octane |
| | valeric acid | | | | | | butyric acid-1-methylpropyl ester |
| | | | | | | | valeric acid-1-methylethyl ester |
| | | | | | | | dibutyl peroxide |
| | | | | | | | valeric acid-propyl ester |
| | | | | | | | valeric acid-1-methylpropyl ester |
| | | | | | | | valeric acid-butyl ester |
| 6 | n-butyric acid/n- | 25/26 | 130 | 68/85 | 58 | 1.149/1.028 | heptane |
| | valeric acid | | | | | | octane |
| | | | | | | | butyric acid-1-methylethyl ester |
| | | | | | | | butyric acid-1-methylpropyl ester |
| | | | | | | | valeric acid-1-methylethyl ester |
| | | | | | | | valeric acid-propyl ester |
| | | | | | | | valeric acid-1-methylpropyl ester |

^{#1}Tests carried out with a working electrode surface of 2.2 cm²

### Example 4

Conversion of a pure carboxylic acid in a continuous flow-through reactor

### Test Structure

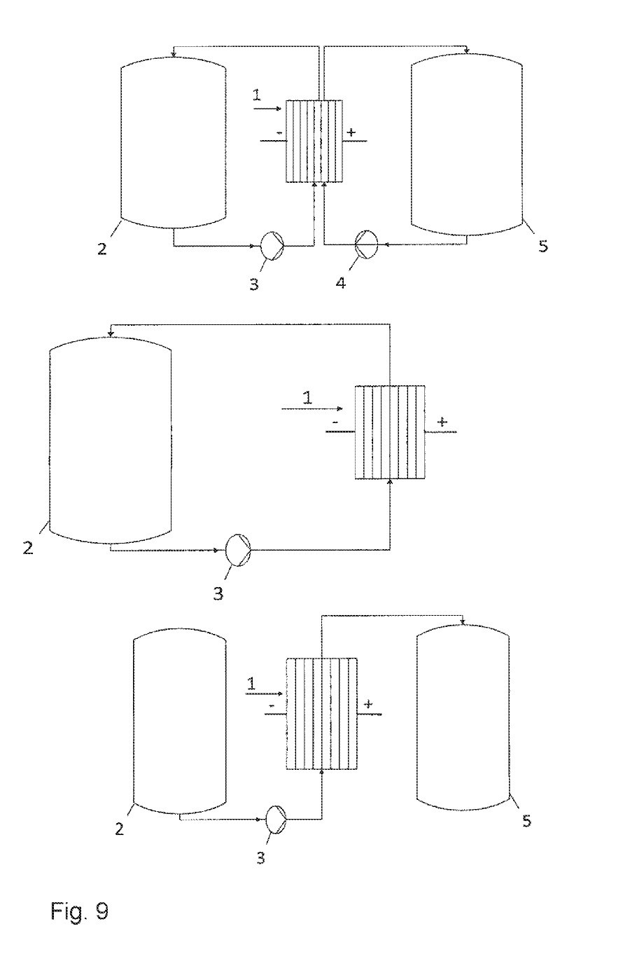

A mixture of carboxylic acids was set to a pH value of 5.5 using potassium carbonate or potassium hydroxide. The tests were carried out in three different configurations in a flow-through reactor (MicroFlowCell, ElectroCell, Denmark) (see top of FIG. 8 and FIG. 9). A platinised titanium electrode having a geometric surface of 10 cm² was used as counter electrode. Either a platinised titanium electrode or a lead electrode with 10 cm² was used as counter electrode. A reference electrode Ag/AgCl 3.4 M KCl was integrated in the system.

The sampling and pH measurements were performed at reservoirs 2 and 5. Depending on the volume flow, a magnetic stirrer was additionally used for continuous mixing of the reaction solution at reservoirs 2 and 5.

### FIG. 9 Test Structure

1: electrochemical cell; 2, 5: reservoir; 3, 4: pump
top: The electrochemical cell 1 consists of an anode chamber and a cathode chamber separated by a membrane. The two electrolyte solutions are each recirculated through a reservoir;
middle: The electrochemical cell 1 is operated without separation of anode chamber and cathode chamber. The reaction solution is pumped around in a circuit from the reservoir 2 with the aid of the pump 3.
bottom: The electrochemical cell 1 is operated without separation of anode chamber and cathode chamber. The reaction solution is pumped around from the reservoir 2 with the aid of the pump 3, the reaction solution is not constantly recirculated. (Note: A combination of the various configurations is possible depending on requirements.)

### Execution

Prior to the test, the tightness of the electrochemical cell was examined. The electrochemical synthesis was then carried out galvanostatically. The reaction time was 0.5 to 8 h depending on reaction conditions. The reaction volume was 10 mL, and the circuit volume varied depending on the test from 25 to 250 mL. Both the voltages between working electrode and counter electrode and between working electrode and reference electrode were recorded (for details see also Table 4). At the end of the test, in addition to the HPLC check of the aqueous phase, the organic phase was also removed and measured by means of GC-MS. Depending on the test, samples of the aqueous phase were also removed during the reaction for control.

### Analytics

The conversion of the carboxylic acid mixture of the aqueous phase was determined by means of HPLC analysis.

An HPLC system (Spectrasystem P4000, Finnigan Surveyor RI Plus Detector, Fisher Scientific, Germany) with a Hyper-REZXP Carbohydrate H+8 mm (S/N:026/H/012-227) column was used. The mobile phase consisted of a 5 mM sulphuric acid solution (flow rate: 0.5 mL/min). The column was cooled to 10° C. For the product concentrations, calibration lines in the range from 0.1 to 100 mM were created. The substances were allocated into corresponding dilutions by their retention time on the basis of previous measurements of standard solutions and were quantified on the basis of the calibration of associated peak areas (R²=0.99).

The formation of the products in the organic phase was determined after the reaction process by means of GC-MS analytics. A GC/MS system (TraceGC Ultra, DSQII, Thermo Scientific, Germany) with a TRWaxMS column (30 m×0.25 mm ID×0.25 μm film GC Column, Thermo Scientific, Germany) or DB-5 column (30 m×0.25 mm ID×0.25 mm film GC Column, Agilent JW Scientific, United States of America) was used.

### Results

TABLE 4
Overview of different galvanostatically performed electrochemical tests with various
carboxylic acids in analogy to the test structure described in Example 4

| | | Starting | Current | | Volume | Circuit | | | |
|---|---|---|---|---|---|---|---|---|---|
| | | concentration | density | Test | flow | volume | Conversion | CE | |
| No. | Substrate | [g/L] | [mA/cm²] | structure | [mL/min] | [mL] | [%] | [%] | Products |
| 1 | n-butyric acid | 22 | 100 | B | 0.7 | 50 | 67 | 19 | hexane (22%) |
| | | | | | | | | | butyric acid-1-methylethyl |
| | | | | | | | | | ester (71%) |
| | | | | | | | | | butyric acid-1-methylpropyl |
| | | | | | | | | | ester (7%) |
| 2 | n-butyric acid | 88 | 100 | B | 0.7 | 50 | 52 | 73 | hexane |
| | | | | | | | | | butyric acid-1-methylethyl |
| | | | | | | | | | ester |
| | | | | | | | | | butyric acid-1-methylpropyl |
| | | | | | | | | | ester |
| 3 | n-butyric acid | 44 | 150 | C | 0.7 | — | 56 | 23 | hexane |
| | | | | | | | | | butyric acid-1-methylethyl |
| | | | | | | | | | ester |
| | | | | | | | | | butyric acid-1-methylpropyl |
| | | | | | | | | | ester |
| 4 | n-butyric acid | 176 | 150 | C | 0.7 | — | 35 | 34 | hexane |
| | | | | | | | | | butyric acid-1-methylethyl |
| | | | | | | | | | ester |
| | | | | | | | | | butyric acid-1-methylpropyl |
| | | | | | | | | | ester |
| 5 | n-butyric acid | 176 | 100 | C | 0.7 | — | 24 | 24 | hexane |
| | | | | | | | | | butyric acid-1-methylethyl |
| | | | | | | | | | ester |
| | | | | | | | | | butyric acid-1-methylpropyl |
| | | | | | | | | | ester |
| 6 | isovaleric acid | 26 | 100 | C | 0.7 | — | 65 | 18 | |
| 7 | n-valeric acid | 51 | 80 | B | 4 | 33 | 95 | 58 | octane |
| | | | | | | | | | dibutyl peroxide |
| | | | | | | | | | valeric acid-1-methylpropyl |
| | | | | | | | | | ester |
| | | | | | | | | | 1-butanol in the aqueous |
| | | | | | | | | | phase |
| 8 | n-valeric acid | 102 | 50 | A | 100 | 250 | 30 | 53 | octane (63%) |
| | | | | | | | | | valeric acid-1-methylpropyl |
| | | | | | | | | | ester |
| 9 | n-valeric acid | 200 | 100 | B | 4 | 50 | 83 | 50 | octane (81%) |
| | | | | | | | | | dibutyl peroxide(3%) |
| | | | | | | | | | valeric acid-1-methylpropyl |
| | | | | | | | | | ester (16%) |
| | | | | | | | | | 1-butanol in the aqueous |
| | | | | | | | | | phase |

1. A method for preparing organic compounds, characterised by
anaerobic fermentation of biomass with mixed microorganism cultures at temperatures of from 10 to 100° C. and pH values of from 3.5 to 9.5, wherein the methane formation is suppressed, such that product liquids comprising mixtures of short-chain and medium length-chain carboxylic acids having a chain length of from 2 to 16 carbon atoms are recovered,
electrolytic treatment of the product liquid containing the carboxylic acids with a constant or varying oxidation current in order to recover the organic compounds,
isolation of the organic compounds.

2. The method according to claim 1, characterised in that for fermentation the biomass is liquid or is brought into contact with a liquid in order to form a fermentation broth and temperatures of from 10 to 100° C. are set, solid fermentation residues are removed as appropriate after fermentation, and the product liquid containing at least 5 g/L of short-chain and medium length-chain carboxylic acids is electrolytically treated, optionally after purification and/or concentration.

3. The method according to claim 1, characterised in that the biomass is selected from at least one of the groups of the energy plants or residual materials and waste products from agriculture and industry, or extracts and processing products therefrom, or algae or yeasts or gas mixtures from biomass gassing or pyrolysis, such as syngas and pyrolysis gas, or silaged biomass or biomass pre-treated by means of other physical, physical-chemical, chemical and/or biological methods.

4. The method according to claim 1, characterised in that the carboxylic acids are in a mixture of branched and/or unbranched mono, hydroxy and/or dicarboxylic acids, preferably carboxylic acids having 4 to 10 carbon atoms.

5. The method according to claim 1, characterised in that the organic compounds comprise, as main products, C₆ to C₁₈ alkanes which are obtained possibly in mixture with corresponding derivatives, such as ethers, esters, alcohols, etc.

6. The method according to claim 1, characterised in that, for fermentation, (a) mixed culture(s) of acid-forming microorganisms and/or methane-forming inhibitor(s) are/is added.

7. The method according to claim 1, characterised in that, for fermentation, alcohols and/or lactic acid are/is added in order to increase the yield of medium length-chain carboxylic acids.

8. The method according to claim 1, characterised in that separate fermenters are used for hydrolysis/acid formation and acetic acid/methane formation, wherein the product liquid from the first-phase reactor is electrolytically treated.

9. The method according to claim 1, characterised in that the product liquid is treated with bases or acids prior to the electrolytic treatment in order to change the pH value.

10. The method according to claim 1, characterised in that the electrolytic treatment is performed with a varying current flow, wherein the current flow (working current flow) is altered at constant or alternating time intervals to another current flow, or what is known as a pulsed flow (pulse method).

11. The method according to claim 10, characterised in that the anode is periodically subjected to an interruption of the power circuit, whereby phases of current flow (production) and no current flow (non production) alternate with one another, wherein the pulse duration varies between 1 second and 2 days, but is always shorter than the duration of the application of the working current flow.

12. The method according to claim 1, characterised in that the fermentation residues created and/or hydrolysis gas formed are used further, preferably for the production of biogas.
